(19) United States
(12) Patent Application Publication
(10) Pub. No.: US 2010/0056878 A1
(43) Pub. Date: Mar. 4, 2010

(54) Indirectly coupled personal monitor for obtaining at least one physiological parameter of a subject

(76) Inventors: Dale L. Partin, Ray Township, MI (US); Michel F. Sultan, Troy, MI (US)

Correspondence Address: Delphi Technologies, Inc., M/C 480-410-202, PO BOX 5052, Troy, MI, 48007, US

(21) Appl. No.: 12/229,932

(22) Filed: Aug. 28, 2008

(51) Int. Cl. A61B 5/00; H04M 11/00

(52) U.S. Cl. 600/301; 379/106.02

(57) ABSTRACT

A method of determining the health status of a subject includes indirectly coupling a personal monitor to at least a portion of a body of the subject, the person monitor including at least one inertial sensor integrated with a mobile telephonic device. The method further includes obtaining data representative of at least one physiological parameter of the subject, and determining the health status based on the data.

### BACKGROUND

The present disclosure relates generally to a method of determining a health status of a subject using a personal monitor.

With aging populations across the world, a need for substantially continuous monitoring of the personal health of members of these populations is noticeably increasing. Many current methods of monitoring the health of a subject employ a personal monitor carried by or otherwise positioned on the subject's body. These personal monitors often use or communicate with one or more sensors configured to obtain data related to various physiological parameters of the subject such as, e.g., heart rate, respiration rate, blood pressure, and the like. In some instances, the sensors are surgically implanted into the subject's body, where output signals from the sensors are sent to an externally-located (relative to the subject's body) monitoring device. In other instances, the sensors are externally coupled to an externally-located monitoring device. In either case, the output signals from the sensors are used to monitor the personal health of the subject.

### SUMMARY

A method of determining the health status of a subject includes indirectly coupling a personal monitor to at least a portion of a body of the subject, the personal monitor including at least one inertial sensor integrated with a mobile telephonic device. The method further includes obtaining data representative of at least one physiological parameter of the subject, and determining the health status based on the data.

### BRIEF DESCRIPTION OF THE DRAWINGS

Features and advantages of the present disclosure will become apparent by reference to the following detailed description and the drawings, in which like reference numerals correspond to similar, though perhaps not identical components. For the sake of brevity, reference numerals or features having a previously described function may or may not be described in connection with other drawings in which they appear.

FIG. 1 is a flow diagram depicting an embodiment of the method of determining a health status of a subject; and

FIG. 2 is a semi-schematic depiction of a personal monitor for use in embodiments of the method disclosed herein.

### DETAILED DESCRIPTION

It has been discovered that inertial sensors may be used to generate signals indicative of various parameters of a subject such as, e.g., for determining a location of the subject and/or for tracking the subject. The inventors of the instant application have unexpectedly and fortuitously discovered that the inertial sensors may also be used to generate a signal related to a physiological parameter of a subject, where such signals may be digitally processed in order to achieve useful physiological information of the subject. As such, embodiment(s) of the method disclosed herein advantageously use a personal monitor having at least one inertial sensor integrated with a wireless communication device to obtain at least one physiological parameter of the subject in a relatively non-intrusive and/or non-invasive manner with respect to the subject's body. In some instances, the physiological parameter(s) may be obtained by the personal monitor even when the monitor is not closely physically coupled to the subject's body. Retention of the personal monitor by the subject may be accomplished a variety of ways without the personal monitor actually contacting the subject's body while still obtaining reliable data of the desired physiological parameter(s). At least some of the methods of retaining the personal monitor by the subject allow the subject to carry the personal monitor without drawing attention to it. The personal monitor may further be used to relay information to the subject and/or to automatically relay or allow the subject to relay information to a third party or a remote device. The personal monitor may also advantageously be used in conjunction with a medical device and may be used to monitor one or more effects of medical treatment applied to the subject.

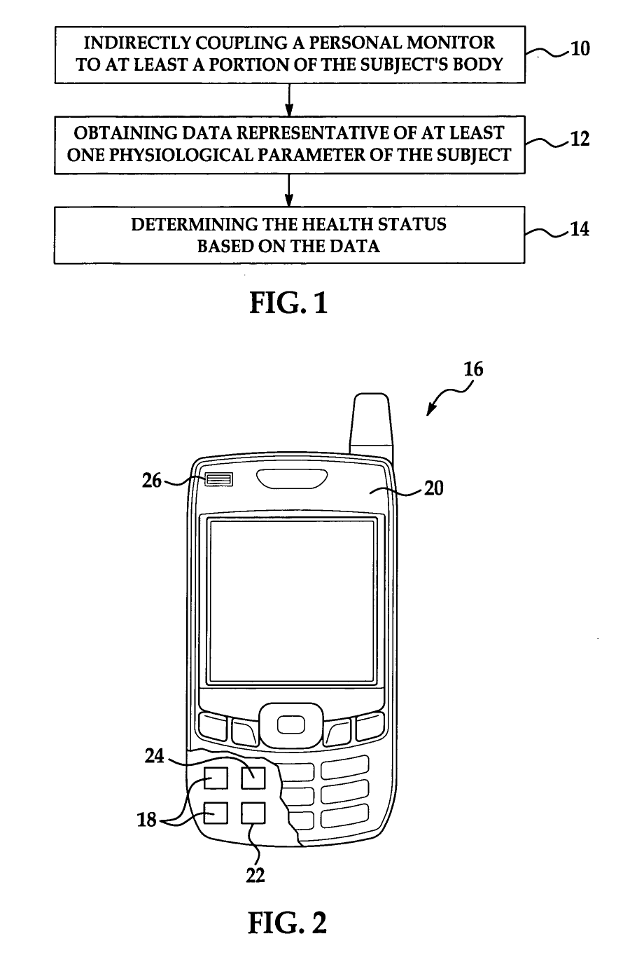

With reference now to FIG. 1, an embodiment of the method of determining the health status of the subject includes indirectly or loosely mechanically coupling the personal monitor to at least a portion of the subject's body (as shown by reference numeral 10), obtaining data representative of at least one physiological parameter of the subject (as shown by reference numeral 12), and determining the health status based on the data (as shown by reference numeral 14).

As used herein, the term “subject” refers to a human being or an animal.

As also used herein, the term “health status” refers to a state of the subject's health on a then-current day and/or at a then-current time of day.

As further used herein, the term “physiological parameter” refers to a vital sign, a physiological element, or the like, non-limiting examples of which include heart rate, respiration rate, a variability in the heart rate, a variability in the respiration rate, physiological impact inflicted on the subject's body, a tremor, a seizure, a cough, activity level (such as, e.g., sitting, running, sleeping, etc.), and/or the like, and/or combinations thereof.

It is to be understood that the term “activity level” refers to a determination that the subject is reasonably active (moves about at least every few hours during normal waking hours, for example). By way of further example, for a subject living alone, a lack of movement over a prolonged period of time (12 hours for example) could lead to a reasonable inference of physiological issue where intervention may be appropriate.

Further, it is to be understood that the term “indirect mechanical coupling” refers to a coupling of the personal monitor via a mechanical linkage associated with the subject's body. For example, a mechanical linkage may be an article worn by the subject such as clothing, jewelry, belts, bands, bracelets, glasses, and/or the like, and/or combinations thereof, and the personal monitor may be connected to the mechanical linkage. When indirect mechanical coupling occurs, signals identifying the physiological parameters originating in the subject's body (e.g., mechanical vibration signals) travels through one or more of the mechanical linkages before the signals are received by the personal monitor. The mechanical linkage may attenuate the mechanical vibration signals.

It is further to be understood that the term “loose mechanical coupling” refers to a coupling of the mechanical linkage (having the personal monitor connected thereto) with the subject's body, where the mechanical linkage is not firmly or rigidly coupled to the subject's body and therefore provides an attenuated signal to the personal monitor. Non-limiting examples of loose mechanical couplings include a belt slideably worn around the subject's waist, a holster clipped to the subject's belt so that the holster may swivel or rotate, a necklace hanging from the subject's neck when the subject is in a bent-over position, and/or the like. With either an “indirect coupling” or a “loose coupling” of the mechanical linkage with the subject's body, the signals corresponding with one or more of the physiological parameters listed above attenuate the signal by a factor ranging from about 5 to about 20 depending upon 1) the distance between the personal monitor and the subject's body, and/or 2) the number and composition of the mechanical linkages between the personal monitor and the subject's body.

The personal monitor used for embodiments of the method depicted in FIG. 1 is semi-schematically shown in FIG. 2. The personal monitor 16 generally includes at least one inertial sensor 18 integrated with a mobile telephonic device 20. In an embodiment, the inertial sensor(s) 18 are disposed inside of the telephonic device 20 (as shown in FIG. 2). While the inertial sensor is disposed inside the telephonic device in this example, it can also be integrated with the exterior of the telephonic device. Disposing the inertial sensor inside the telephonic device generally makes it easier to provide electrical power to the sensor, to get signals from the sensor, and to protect the sensor from environmental factors such as moisture. The mobile telephonic device 20 is depicted in FIG. 2 as a cellular phone. It is to be understood, however, that the mobile telephonic device 20 may be any suitable wireless communication device, non-limiting examples of which include, in addition to a cellular phone, a pager, a personal digital assistant (PDA), a fob and/or the like.

The inertial sensor(s) 18 are generally selected from devices configured to generate a signal (often measurable as a voltage that may be related to acceleration in units of, e.g., m/s² or g's) for one or more physiological parameters of the subject when the personal monitor 16 is indirectly coupled to at least a portion of the subject's body. Non-limiting examples of suitable devices for the sensor(s) 18 include a single-axis accelerometer, a multi-axis accelerometer, a single-axis gyroscope, a multi-axis gyroscope, a tilt sensor, a vibration sensor, and/or the like, and/or combinations thereof. There are several different ways or techniques that may be used to convert data generated by the signal into the appropriate units for the physiological parameter. Details of an example of a method of using data to determine, e.g., the subject's heart rate may be found in U.S. Publication Number 2005/0022606, filed Jul. 31, 2003, which is commonly owned by the Assignee of the instant Application and is herein incorporated by reference in its entirety.

It is to be understood that, since the personal monitor 16 is indirectly coupled to the subject's body, the exact magnitude of the signal may not be, in some instances, easily ascertainable. In these instances, the size of the signal may be used to determine the desired physiological parameter. For example, if respiration rate or heart rate is desired, a sudden, larger signal than expected may indicate that the subject is engaged in a particular activity such as, e.g., running, stair climbing, or the like. Other signals related to, e.g., coughing and falling may be deduced from the amplitude, frequency, a characteristic acceleration signal (if one is available), and/or the like.

In a non-limiting example, the mobile telephonic device 20 includes an electronic circuit board (not shown) and/or other electronic components substantially rigidly attached to the device 20. The inertial sensor(s) 18 may, in an embodiment, be attached to the circuit board and/or the other electrical components via, e.g., soldering or other suitable metallurgical attachment means. Any mechanical vibration, tilt, rotation, or the like endured by the device 20 is readily transferred to the inertial sensor(s) 18.

For purposes of illustration, the personal monitor 16 shown in FIG. 2 has two inertial sensors 18 integrated in the mobile telephonic device 20. It is to be understood, however, that any number of inertial sensors 18 (e.g., one, two, three, four, or more) may otherwise be used. For example, the personal monitor 16 may include two sensors 18, where one sensor 18 is configured to generate a signal representative of, e.g., acceleration in the plane of the circuit board in two generally perpendicular directions, whereas the other sensor 18 may generate a signal representative of acceleration in a direction perpendicular to the circuit board.

It is to be understood that reasonable resolution of the inertial sensors 18, as well as the signal processing is desirable. “Reasonable resolution” as used herein is defined as 12 bit, 16 bit, or more. In a non-limiting embodiment, an accelerometer may be used with a span of +2 g, and an amplifier with a gain of 10 or 20 may be used to amplify the accelerometer output before it is digitized for analysis of respiration and/or cardiac signals. In an example, a digital filter with a band pass of 0.2 Hz to 0.8 Hz may be used to isolate the respiration signal; and a digital filter with a band pass of 0.8 Hz to 30 Hz may be used to isolate the cardiac signal. It is to be understood that analog filters with the same frequency ranges may be used before digitization.

It is to be further understood that the 0.8 Hz “break” frequency used in the example above to separate the respiratory and cardiac frequency bands may be adjusted for individual differences. For example, adjusting the break frequency to, e.g., 0.5 Hz may be more suitable for a subject with relatively low respiration and heart rates.

The signal may optionally be squared after filtering. The power spectral density (PSD) of the signal may then be computed. The PSD spectrum will have a major peak at the respiration frequency or at the cardiac frequency, depending upon which frequency band was used. Harmonics of the cardiac and respiratory frequencies may normally also be present.

The personal monitor 16, including its several internal components such as, e.g., the circuit board, etc., may be powered by any suitable means. If, e.g., the personal monitor 16 is being used as a stationary device, an electrical power means may be used. In addition to or in the alternative, the personal monitor 16 may be powered via a battery, a solar cell, or the like. Such powering means is particularly advantageous in situations in which the personal monitor 16 may be carried by the subject. Further, in instances where a battery is used, the personal monitor 16 may also have an energy-scavenging device configured to recharge the battery. Also, the personal monitor 16 may be configured to conserve power, especially when the monitor 16 is operated on a battery. Conservation of power may be achieved by, e.g., operating the sensor(s) 18 on a relatively low duty cycle (e.g., about 1% to about 10%), which corresponds to a sensor bandwidth ranging from about 1 Hz to about 10 Hz. For example, since respiration generally occurs at a rate of less than 2 Hz, operating the sensors 18 at higher frequencies is unnecessary. Sometimes, however, use of the entire range of the bandwidth may be necessary. For example, since harmonics of a subject's heart rate may be important, using a sensor bandwidth of up to about 30 Hz may be useful. In some instances, a sensor bandwidth of more than 30 Hz may otherwise be used.

In an embodiment, the personal monitor 16 further includes an audio device 26 (schematically depicted in FIG. 2). In an example, as shown in FIG. 2, the audio device 26 may be integrated with the telephonic device 20. In another example (not depicted in the figures), the audio device 26 may be separate from, yet operatively connected to, the telephonic device 20. As will be described in further detail below, the audio device 26 is generally configured to allow the subject to receive audible or even verbal notifications of his/her current physiological status including any potential physiological problems detected by the personal monitor 16.

In an embodiment, the personal monitor 16 may be indirectly coupled to the subject's body by retaining the personal monitor 16 in, or otherwise connecting the personal monitor 16 to an article worn by the subject. Non-limiting examples of articles include garments, bands (such as a wrist or ankle band, bracelet, or the like), belts, and/or the like, and/or combinations thereof. Indirect contact between the personal monitor 16 and the subject's body may also be accomplished by placing and at least temporarily retaining the personal monitor 16 in a holster, whereby the holster is configured to be worn by the subject. Other examples of indirect contact involves connecting the personal monitor 16 to a piece of jewelry such as, e.g., to a pendant on a necklace or to a pair of eyeglasses. In yet another example, the monitor 16 may indirectly contact the subject by operatively connecting the personal monitor to at least one medical device (e.g., a medical pump, an oxygen-generating device, and/or another similar device), where the medical device is operatively connected to the subject's body. It is to be understood that any indirect coupling of the personal monitor 16 to the subject's body may be accomplished without using an electrical or optical coupling means that, e.g., an electrocardiogram or a pulse oximeter would otherwise require.

In some instances, the subject may automatically be asked, by the personal monitor 16, to place the personal monitor 16 in a particular position in response to detected abnormal conditions of the subject. In such instances, the personal monitor 16 may be temporarily placed directly adjacent the subject's body to obtain more definitive data related to the desired physiological parameter. For example, if signals related to the subject's heart rate are desired, the subject may be asked (as described in more detail below) to place the personal monitor 16 against his/her chest (typically through the subject's clothing), substantially adjacent to the subject's heart. One or more signals indicative of the subject's heart rate may be generated.

It is to be understood that the personal monitor 16 may be configured for the manner for which the monitor 16 will be or is intended to be used. For instance, if the personal monitor 16 is retained in a holster, it may be desirable to use, e.g., a cellular phone, as a suitable telephonic device 20. If, on the other hand, the personal monitor 16 is connected to a pendant on a necklace, it may otherwise be desirable to use, e.g., a fob as the telephone device 20. It is further to be understood that any configuration may be used in any application as desired by the subject and/or by a user of the personal monitor 16 (such as, e.g., a clinician or nurse).

In an embodiment, as also shown in FIG. 2, the personal monitor 16 further includes a locating device 22 such as, e.g., a GPS or other triangulation device. As will be described in more detail below, the locating device 22 may be used, e.g., by a third party or a remote device to determine the location of the personal monitor 16 at a then-current day and/or a then-current time of day.

It is to be understood that, in some instances, the personal monitor 16 may desirably be coupled to the subject's body at all times. For these situations, the personal monitor 16 may be configured to be used in a variety of different environmental conditions including, e.g., rain, heat, humidity, freezing temperatures, and/or the like. It is to be understood that the inertial sensor(s) 18 selected for the personal monitor 16 are generally already configured (via the manufacturer) to handle such variations in environmental conditions. In some instances, the personal monitor 16 may further be configured to be waterproof so that the personal monitor 16 may be used when the subject is taking a shower. Waterproofing may be accomplished simply by the integration of the sensor(s) 18 inside the telephonic device 18 (such as shown in FIG. 2), or via other methods sufficient to substantially prevent the sensor(s) 18 from being exposed to the external environment.

In a non-limitative example, signals generated by the sensor(s) 18 indicative of one or more physiological parameters of the subject are obtained by the personal monitor 16 and stored as data in, e.g., an electronic memory associated with the personal monitor 16. In an example, the electronic memory is integrated in the mobile telephonic device 20. In this example, the signals are automatically analyzed by the personal monitor 16 using an electronic processing device internal to the monitor 16. The results from the analysis may thereafter be transmitted to the subject, to a third person, and/or to another device (e.g., a personal computer) via wireless and/or audio methods. It is to be understood that the signals may be automatically analyzed continuously, periodically, and/or when a previously analyzed result indicates a potential physiological problem.

In another example, the electronic memory is located in another device (e.g., a personal computer) located remotely from the personal monitor 16. In this example, the signals generated by the sensor(s) 18 are automatically or manually communicated, via the telephonic device 20 using audio and/or wireless methods, to the remote device. The data may be used either by the subject, a third party (e.g., an authorized person, a caregiver, or the like), or even a remote device (e.g., a computer, an alarm system, or the like) to determine the then-current health status of the subject.

The personal monitor 16 is generally operated to substantially continuously generate signals indicative or representative of one or more physiological parameters of the subject. In an embodiment, the monitor 16 may be operated so that data is obtained at various rates. For example, the rate may be, e.g., about twenty times per second, or every five milliseconds when the personal monitor 16 is in physical contact with the subject. In another example, the rate may be, e.g., about once every few minutes when the personal monitor 16 is not in physical contact with the subject.

In instances where there is no activity and/or no signals being generated by the personal monitor 16 over a predetermined amount of time, the monitor 16 may enter a power-saving mode. In the power-saving mode, the monitor 16 is periodically activated to check whether indirect contact with the subject has been restored and/or whether a physiological parameter is desired. When at least some activity is detected, the monitor 16 resumes its original operational state.

In an embodiment, the data related to one or more physiological parameters of the subject may also be obtained by, e.g., a nurse, a clinician, or another third party by relaying a communication to the subject via the personal monitor 16 requesting the data. The third party may communicate with the personal monitor 16 by placing a call therewith, sending a text message thereto, sending an alert thereto to trigger one or more indicators, and/or the like, and/or combinations thereof. Non-limiting examples of indicators include a vibration of the personal monitor 16, one or more blinking lights, an audible trigger, e.g., a predefined sound such as a beep, and/or the like, and/or combinations thereof. If the subject is available, the indicator will alert the subject that data has been requested.

In response to the request, the subject may send or otherwise transmit a reply to the request. In a non-limiting example, the reply may be a verbal reply, an audible reply, and/or a visual reply to the third party. If the subject uses an audible reply, the audible reply may be a sequence defining the data, similar to a Morse Code. In another non-limiting example, the subject may reply via a data transmission from the personal monitor 16 to a remote device (e.g., a computer). If a data transmission is used, the data may be transmitted manually by the subject by, e.g., entering the data on a keypad or other user interface of the personal monitor 16. A transmission function may then be executed on the personal monitor 16. If however, the subject is not available, the requested data may be automatically transmitted from the personal monitor 16 to the remote device upon request by either the third party or the remote device.

It is to be understood that the remote device may also relay a communication with the personal monitor 16. In an example, the remote device may be programmed to periodically send requests for data to the personal monitor 16. In another example, a third party may send an individual request for data. This may be particularly useful in situations in which the third party suspects that the subject's health may be deteriorating.

In another embodiment, the communication relayed to the personal monitor 16 includes a request for data regarding to how the subject feels. For example, a third party may call or send a text message to the personal monitor 16 asking the subject how he/she is feeling at the then-current day and/or the then-current time of day. The subject may verbally respond to the request if the request was made via a telephone call, may respond by sending a text message, and/or may respond via any other suitable communication means. In this embodiment, the subject may respond by assuring that he/she does or does feel well.

In some instances, the subject may be requested to perform an action such as, e.g., to take a measurement of a specific physiological parameter. If the data related to the specific physiological parameter indicates that the parameter is outside a predefined range, the subject may be asked to perform a specific action in order to obtain a clearer reading of the physiological parameter. For example, the subject may be asked to place the monitor 16 against his/her chest for a clearer reading of the subject's heart rate (as indicated above). In another example, the subject may be asked to take a measurement while the subject is engaged in a particular activity (e.g., while the subject is walking). The new measurement may be used to check how much the data representative of the physiological parameter has changed compared to an average value taken over a period of time. The comparison information may then be used to determine if there are any significant changes to the subject's health over time.

Prior to making a data request, the subject may also be asked to verify that he/she is a correct subject. Verification may be accomplished, e.g., by asking the subject at least one verification question such as “What is your name?”, “What is your mother's maiden name?”, “What is the model of your first car?”, “What is your first pet's name?”, and/or the like. Another way of verifying the subject includes requesting the subject to enter a numerical code or password using the user interface associated with the personal monitor 16. A correct response to one or more of the questions or a correct code or password would verify that the subject answering the question(s) or request is the correct subject.

For any of the embodiments set forth above, the personal monitor 16 may further be configured to determine an appropriate response or action based on the health status of the subject. The response or action is generally based on the data representative of one or more physiological parameters of the subject. The response may include, but is not limited to, a diagnosis of a false indication of a problem or condition, alerting the subject of a potential problem, instructing the subject for further diagnosing the potential problem to determine if a problem does in fact exist, automatically applying a therapeutic action for the subject, alerting emergency responders and/or other third parties that a problem exists, and/or the like, and/or combinations thereof.

In a non-limiting example, the response or action is determined by the personal monitor 16 via an algorithm. For example, measurements of the subject's heart rate may be taken while the subject is at rest to establish a base heart rate. If, e.g., a significantly elevated heart rate is later detected and/or measured, a physiological problem may be present unless, e.g., the subject is engaged in an activity that necessitates a higher heart rate. In the event that the subject's heart rate falls back to the base heart rate after a period of time (e.g., a few minutes), the heart rate event may not be significant. If, on the other hand, the heart rate remains elevated, is elevated while engaged in a resting activity, and/or exceeds a predetermined threshold level, then the heart rate may be significant, thereby indicating a potential problem. The algorithm may include a similar protocol for other desired physiological parameters of the subject.

In an example, the algorithm also includes a learning feature, where the personal monitor 16 learns positive indications of a current or potential health problem using information accumulated by the monitor 16 over a period of time. The personal monitor 16 uses and analyses the data to learn an indication of a potential health problem for future use. It is to be understood that the analysis and/or diagnosis of a problem or condition may be performed by the personal monitor 16 or via wireless communication with a remote device. In instances where a remote device is used, the remote device is programmed with the algorithm to determine the appropriate response or action.

Additional steps may also be taken to remove or otherwise filter out false indications of a problem before contacting a third party or an emergency responder. If a potential problem is detected using the data analysis, the personal monitor 16 may automatically alert the subject about the detected potential problem. At this point, the personal monitor may request an assurance from the subject that the subject is fine. If such an assurance is made, no further action is taken unless an analysis of the data indicates otherwise. If, however, the subject responds with a negative indication of his/her health or fails to respond to the alert, then a message may be wirelessly sent to a third party or to an emergency responder.

In the event that there is a potential problem and the subject is not sure whether the potential problem is real, the subject may be asked to take certain actions to further diagnose the problem. For example, the subject could be asked to hold the monitor 16 against his/her chest in various places to assess the subject's heart condition (similar to a stethoscope). In another example, the monitor 16 could be provided with an electrocardiogram (ECG) test feature. In this example, the subject may be asked to hold one part of the monitor 16 with one hand and to touch another part of the monitor 16 with the other hand. The ECG may be measured by the difference in potential between the subject's hands. In yet another example, the personal monitor 16 may include a microphone (not shown) to determine whether the subject is, e.g., coughing excessively.

In the embodiment where the personal monitor 16 is operatively connected to a medical device, the medical device may be activated in the event that the personal monitor 16 indicates that one or more physiological parameters are abnormal. For example, if the personal monitor 16 determines that the subject's respiration rate is too high, and the personal monitor 16 is operatively connected to an oxygen generating device (not shown), the oxygen generating device may be activated and instructed (by the personal monitor 16) to supply an appropriate amount of oxygen to the subject until the subject's respiration rate normalizes. Another example of a medical device is an infusion pump (not shown) which may be activated, deactivated, or otherwise controlled in order to substantially maintain a relevant physiological parameter within a normal/desired range.

It is to be understood that the personal monitor 16 may also be used by the subject as a means for communicating a distress signal to a third party during an emergency situation such as, e.g., if the subject is having a heart attack, has fallen down, and/or the like. In these situations, the subject may initiate the communication with the third party, via the personal monitor 16, to request assistance. In a non-limiting example, the communication may be initiated by the third party by tapping the personal monitor 16 in a sequence defining the request. The taps are then transmitted to a remote device, which translates the coded message to a human-readable alert message. Depending on the severity of the alert message, the subject may be contacted by a trained medical assistant with first aid and/or other medical instructions for the subject, or emergency personnel may be dispatched to the subject's location. In the latter situation, the location of the subject may be determined using the locating device 22 in the personal monitor 16.

It is to be understood that other coded messages may be used by the personal monitor 16 for reasons other than for emergency situations. For example, the subject may tap the personal monitor 16 in a coded sequence for silencing the monitor 16 if the telephonic device 18 portion of the monitor 16 rings, or the like.

In some instances, a third party may want to check up on the subject when the subject is, e.g., sleeping. In this case, the third party may be given permission to contact the personal monitor 16 and to receive data related to the subject's current health state, a history of recent activity level, any irregularities of one or more physiological parameters of the subject, and/or the like. The data may be received by the third party in an audible or data format directly from the personal monitor 16, or electronically through, e.g., a website. In any event, before the third party may receive the information, the third party is verified via any of the verification means provided above.

In other instances, a third party may want to contact the subject via, e.g., a telephone call. If the subject happens to be sleeping at the time the call is made, the personal monitor 16 may be configured to notify the caller that the user is in fact asleep. When the personal monitor 16 determines that the subject is awake (by, e.g., an increase in heart rate, or other indication of at least some activity of the subject), the personal monitor 16 may further be configured to notify the caller that the subject is currently awake. Notification may be accomplished via a variety of ways such as, e.g., automatically sending a call to the caller indicating that the subject is awake. In another non-limiting example, the personal monitor 16 may also be configured to allow the caller to change the ringtone of the telephonic device 18 of the personal monitor 16 to or from silence, vibrate, and ringer so that the subject will not be disturbed when the subject is sleeping or to contact the subject when the subject is awake.

It is to be understood that the effectiveness of the personal monitor 16 to determine the health status of the subject is dependent on whether the personal monitor 16 consistently provides accurate data. To assure that relatively accurate results are obtained from the personal monitor 16, the personal monitor 16 may be calibrated as often as appropriate. In an embodiment, calibration may be accomplished manually via the subject, a technician, or another third party. In another embodiment, calibration may be accomplished remotely. Remote calibration may be achieved by actuating (by, e.g., the technician) a vibrating actuator 24 (shown in FIG. 2) operatively connected to the personal monitor 16. Upon actuation, the vibrating actuator 24 begins to vibrate, thereby generating a vibration frequency and an amplitude. The vibration frequency and the amplitude is read by the inertial sensor(s) 18, which responds to the vibration by generating one or more output signals reflecting the vibration frequency and the amplitude. The output signal(s) (i.e., data) are communicated back to the technician using the telephonic device 20. The technician, e.g., compares the data with a predetermined value to determine if the monitor 16 needs to be calibrated. If the response is substantially similar to the predetermined value, then the personal monitor 16 is considered to be functioning within calibration limits. If, however, the response is not similar to the predetermined value, then the personal monitor 16 is not considered to be functioning within calibration limits. In this case, the subject may be alerted indicating that the monitor 16 should be serviced.

It is to be understood that the terms “connect/connected,” “couple/coupled” or the like is broadly defined herein to encompass a variety of divergent connecting or coupling arrangements and assembly techniques. These arrangements and techniques include, but are not limited to (1) the direct connection or coupling between one component and another component with no intervening components therebetween; and (2) the connecting or coupling of one component and another component with one or more components therebetween, provided that the one component being “connected to” or “coupled to” the other component is somehow operatively coupled to the other component (notwithstanding the presence of one or more additional components therebetween).

While several embodiments have been described in detail, it will be apparent to those skilled in the art that the disclosed embodiments may be modified. Therefore, the foregoing description is to be considered exemplary rather than limiting.

What is claimed is:

1. A personal monitor, comprising:
a mobile telephonic device; and
at least one inertial sensor integrated with the mobile telephonic device;
wherein the personal monitor is configured to obtain at least one physiological parameter of a subject when the personal monitor is indirectly coupled to at least a portion of a subject's body.

2. The personal monitor as defined in claim 1 wherein the mobile telephonic device is selected from a cellular phone, a pager, a personal digital assistant, a fob, and combinations thereof.

3. The personal monitor as defined in claim 1 wherein the at least one physiological parameter is selected from heart rate, respiration rate, a variability of the heart rate, a variability of the respiration rate, physical impact inflicted on the subject's body, a tremor, a seizure, a cough, an activity level, and combinations thereof.

4. The personal monitor as defined in claim 1, further comprising a locating device configured to locate the personal monitor at a then-current day, a then-current time of day, a then-current geographic position, or combinations thereof.

5. The personal monitor as defined in claim 1 wherein the at least one inertial sensor is selected from a single-axis accelerometer, a multi-axis accelerometer, a single-axis gyroscope, a multi-axis gyroscope, a tilt sensor, a vibration sensor, and a combination thereof.

6. The personal monitor as defined in claim 1, further comprising an audio device integrated with or operatively connected to the mobile telephonic device, wherein the audio device is configured to at least provide audible messages to the subject, verbal messages to the subject, or combinations thereof.

7. The personal monitor as defined in claim 1 wherein the personal monitor is configured to be operatively connected to at least one medical device.

8. A method of determining a health status of a subject, comprising:
indirectly coupling a personal monitor to at least a portion of a body of the subject, the personal monitor including at least one inertial sensor integrated with a mobile telephonic device;
obtaining data representative of at least one physiological parameter of the subject; and
determining the health status based on the data.

9. The method as defined in claim 8 wherein the personal monitor is indirectly coupled to at least a portion of the subject's body by operatively connecting the personal monitor to a medical device, wherein the medical device is operatively connected to the subject's body.

10. The method as defined in claim 9, further comprising:
instructing the medical device to perform a predetermined therapeutic action; and
performing the predetermined therapeutic action.

11. The method as defined in claim 8 wherein the personal monitor is indirectly coupled to at least a portion of the body of the subject by retaining the personal monitor in an article worn by the subject.

12. The method as defined in claim 11 wherein the article is at least one of a holster, a garment, a band, a belt, a piece of jewelry, a pair of eyeglasses, or combinations thereof.

13. The method as defined in claim 8 wherein the obtaining the data of the subject is accomplished by:
relaying a communication to the subject via the personal monitor; and
transmitting a reply from the subject in response to the communication if the subject is available, the reply being transmitted to a third party, a remote device, or a combination thereof.

14. The method as defined in claim 13 wherein the communication is a request for information relating to the at least one physiological parameter of the subject, an inquiry of how the subject feels, a request that the subject perform an action, and combinations thereof.

15. The method as defined in claim 14 wherein the request or inquiry is made by at least one of a verbal message, text message, a predefined sound, a vibration, or combinations thereof.

16. The method as defined in claim 14 wherein the action is selected from taking a measurement of the at least one physiological parameter, placing the personal monitor substantially adjacent to a selected portion of the subject's body, engaging in an activity in order to obtain an updated measurement of the at least one physical parameter, an assurance that the subject does or does not have a problem, verifying that the subject is a correct subject, and combinations thereof.

17. The method as defined in claim 13 wherein the reply from the subject is a verbal reply, an audible reply, a visual reply, and combinations thereof.

18. The method as defined in claim 17 wherein the audible reply is a sequence defining the data.

19. The method as defined in claim 8 wherein the obtaining the data is accomplished by:
relaying a communication to the subject via the personal monitor; and
automatically transmitting, to a remote device, a reply from the personal monitor in response to the communication if the subject is not available.

20. The method as defined in claim 19, further comprising transmitting, from the remote device, a communication to a third party requesting assistance for the subject.

21. The method as defined in claim 19, further comprising automatically applying, via the personal monitor, a therapeutic action for the subject.

22. The method as defined in claim 19 wherein the reply includes the at least one of a physiological parameter of the subject or a location of the personal monitor.

23. The method as defined in claim 8, further comprising:
communicating, after determining the health status of the subject, a response to the subject, a third party, or a combination thereof, wherein the response is based on the data of the condition and is selected from an alert of a potential problem, one or more instructions for further diagnosis if a problem exists, one or more instructions for a therapeutic action, and combinations thereof.

24. The method as defined in claim 23 wherein prior to communicating the response, the method further comprises:
accumulating data pertaining to the at least one physiological parameter of the subject over a period of time;
determining an indication of a potential health problem from an analysis of the data; and
learning, via the personal monitor, an indication of the potential health problem for future use.

25. A method of requesting assistance using a personal monitor, comprising:
indirectly coupling a personal monitor to at least a portion of the subject's body, the personal monitor including one or more accelerometers integrated with a mobile telephonic device;
initiating, by the subject, a request for the assistance by tapping the personal monitor in a sequence defining the request; and
transmitting the request to a remote device.

26. A method for calibrating a personal monitor, comprising:
actuating a vibrating actuator operatively connected to the personal monitor, the personal monitor including an accelerometer integrated in a mobile telephonic device;
reading and responding to, via the accelerometer, a vibration frequency and amplitude from the vibrating actuator; and
comparing the response to a predetermined value;
wherein, if the response is substantially similar to the predetermined value, then the personal monitor is considered to be functioning within calibration limits.
